(19) United States
(12) Patent Application Publication
(10) Pub. No.: US 2013/0211071 A1
(43) Pub. Date: Aug. 15, 2013

(54) METHOD FOR MAKING ALPHA-AMINO-EPSILON-CAPROLACTAM USING MIXED SUPER CRITICAL FLUIDS

(71) Applicants: William Rusty Sutterlin, Hoover, AL (US); Mark G. Tegen, Gig Harbor, WA (US); William W. Berry, Lakeland, FL (US)

(72) Inventors: William Rusty Sutterlin, Hoover, AL (US); Mark G. Tegen, Gig Harbor, WA (US); William W. Berry, Lakeland, FL (US)

(73) Assignee: INVENTURE RENEWABLES, INC., Tuscaloosa, AL (US)

(21) Appl. No.: 13/825,799

(22) PCT Filed: Sep. 23, 2011
(86) PCT No.: PCT/US11/53125

§ 371 Date: Apr. 18, 2013

(30) Foreign Application Priority Data
Sep. 24, 2010 (US) 61386433

(51) Int. Cl. C07D 223/10; C07D 223/12

(52) U.S. Cl. CPC C07D 223/10; C07D 223/12
USPC 540/528; 540/533

(57) ABSTRACT

The present invention can involve a method of synthesizing α-amino-ε-caprolactam. The method can comprise heating a salt of L-lysine in a solvent comprising an alcohol under Super Critical Fluid conditions. The methods can comprise heating a salt of L-lysine in a solvent comprising an alcohol and deaminating the reaction product. In various embodiments, the invention can include methods of converting biomass into nylon 6. The methods can comprise heating L-lysine in a solvent comprising an alcohol to produce α-amino-ε-caprolactam, deaminating to produce ε-caprolactam and polymerizing into nylon 6, wherein the L-lysine is derived from biomass. In other embodiments, the present invention can include methods of making nylon 6. The methods can comprise synthesizing ε-caprolactam and then polymerizing, wherein the ε-caprolactam is derived from L-lysine.

### CROSS REFERENCE TO RELATED APPLICATIONS

This application claims the benefit. of U.S. Provisional Patent Application Ser. No. 61/386,433 filed on Sep. 24, 2010, which is incorporated herein by reference,

### FIELD OF THE INVENTION

The present invention relates to a method of synthesizing a caprolactam, and more specifically, synthesizing ε-caprolactam from L-lysine.

### BACKGROUND OF THE INVENTION

About 2.5 billion tons of nylon 6 is produced annually on a worldwide basis. The production of nylon 6 is accomplished by the ring opening polymerization of the monomer it ε-caprolactam. The starting chemical compound for the production ε-caprolactam is benzene which is converted to either cyclohexane or phenol and either chemical is converted via cyclohexanone to cyclohexanone oxime and then this intermediate is heated in sulfuric acid. This chemical reaction is known as the Beckman rearrangement. The starting chemical benzene is produced via the refinement of petroleum chemicals.

### SUMMARY OF THE INVENTION

The inventors herein have succeeded in devising a new approach in the production of ε-caprolactam from, natural products. The approach is based upon the use of L-lysine in a novel process to produce ε-caprolactam which is needed as precursor to nylon 6.

Thus, in various embodiments, the present invention provides a method of synthesizing α-amino-ε-caprolactam, comprising heating a salt of L-lysine in a solvent comprising an alcohol. In various embodiments, the methods comprise heating to stilt of L-lysine in a solvent comprising an alcohol, and deaminating the reaction product. In various embodiments, the invention includes methods of converting biomass into nylon 6. Such methods comprise beating L-lysine in a solvent comprising an alcohol to produce α-amino-ε-caprolactam, deaminating to produce ε-caprolactam and polymerizing into nylon 6, wherein the L-lysine is derived from biomass.

### DETAILED DESCRIPTION OF THE INVENTION

Caprolactam is primarily used in the manufacture of synthetic fibers, especially nylon 6 that is also used in bristle brushes, textile stiffeners, film coatings, synthetic leather, plastics, plasticizers, vehicles, cross linking for polyurethanes, and in the synthesis of lysine. The starting point for the production of ε-caprolactam is benzene which is refined from the non-renewable source of petroleum. In addition to its limitations due to its source of non-renewable petroleum, exposure to benzene, which has been linked to acute myeloid leukemia and non-Hodgkin's lymphoma, is a continuing problem for the chemical industry. The most effective way of dealing with benzenes human health risk is to eliminate its use.

The cyclization of L-lysine to form a seven member ring of α-amino-ε-caprolactam has been attempted before and reports have shown low yields. Such attempts have included reactions in near super critical water (see Japanese Patent No. 2003206276 to Goto et al. issued Jul. 22, 2003) or reactions using an excess of Al₂O₃ in toluene (see Blade-Font, A., Tetrahedron Lett., 1980, 21, 2443-2446. Pellegata, R., Pinza, M.; Pifferi G., Synthesis 1978, 614-616).

In one aspect, the invention provides an efficient route for the cyclization for a cyclic amidation reaction to form lactams having ring sizes from 5 to 8 ring members. Following cyclic amidation, other reactive groups on the cyclic ring may be removed if desired. In one aspect, the invention provides efficient cyclic amidation carried out in an alcohol solvents having from 2 to 6 carbons. Amino functional carboxylic acid useful in the invention improves those that can cyclize to form a stable lactam, preferably one having from 5 to 8 ring members. The amino functional carboxylic acids can contain other functional groups as long as those functional groups do not interfere with the amidation reaction mediated by the 2 to 6 carbon alcohol solvent.

According to the present invention, a new process for the cyclization of L-lysine to α-amino-ε-caprolactam is described herein. In addition, in accordance with the present invention, a process for the domination of α-amino-ε-caprolactam to ε-caprolactam is described herein. Commercially available sources of L-lysine such as, but not limited to, L-lysine dihydrochloride, L-lysine hydrochloride, L-lysine, phosphate, L-lysine diphosphate, L-lysine acetate, and L-lysine may be used and any ceded steps so that the L-lysine is in the proper state for the following reactions will be known by one skilled the art. In addition, commercially available sources of lysine maybe used but a step to separate the L-lysine from the alysine may be added such as, for an example, a chiral separation step and such separation and purification. techniques will be known by one skilled in the art. In various embodiments, a cyclization reaction was initiated without the need for neutralization of lysine hydrochloride with sodium hydroxide (NaOH). In this embodiment, none of the then resulting NaCl would need to be precipitated out of the solution. In various embodiments, water that is generated during the cyclization reaction does not need to be removed as the reaction occurs in the super critical state and any small amounts of water generated axe moved to the as phase by super critical partial pressure and do not affect the cyclization reaction. No water removal is necessary to provide for the reaction to occur.

Non-limiting examples of alcohols include 1-propanol, 2- propanol, 1-butanol, 2-butanol, isobutanol, 1,2-propanediol, 1,3-propanediol, 1,2-butanediol, 1,4-butanediol, all isomers of 5 carbon monols, diol and triols including with out limitation 1-pentanol, 1,2-pentanediol, 1,5-pentanediol, and all isomers of 6 carbon monodiols, diols and trials including without limitation, 1-hexanol, 1,2-hextanediol, 1,6-hexanediol. Other nonlimiting examples of 2 to 6 carbon alcohols include glycerol, trimethylolpropane, pentaerythritol and the like. In various embodiments, the alcohols have a single hydroxyl group. in other embodiments, the alcohols have 2 hydroxyl groups. In some embodiments, the alcohols have 3 hydroxyl groups. Nonlimiting examples of glycols include propylene glycol, butylene glycol, neopentyl glycol and the like.

Biomass used can include lysine hearing amino acids such as algae, cyanobacteria, yeast, jatropha, soy bean, canola beans, rapeseed and other protein rich biomass.

### EXAMPLES

### Example 1

A mixture a hydrochloride 1 (100 g) methanol (200 g) is heated to 280 C and 1800 psi and held at temperature and pressure for 1 hour. The resulting material is analyzed as crude α-amino-ε-caprotactamin X % yield.

1. A process for synthesizing a α-amino -ε-caprolactam, the process comprising:
heating a salt of lysine in a solvent comprising: an alcohol, a water or an alcohol and a water, without the presence of a catalyst, at a temperature of about 235° C. to about 320° C., to produce the α-amino-ε-caprolactam.

2. The process of according to claim 1, further comprising:
(a) purifying the α-amino-ε-caprolactam; or
(b) crystallizing the α-amino-ε-caprolactam.

3. (canceled)

4. The process of claim 1, wherein the lysine is L-lysine.

5. The process of claim 1, wherein the alcohol has from 2 to 6 carbons.

6. The process of claim 1, wherein the alcohol comprises a diol, a triol, a glycol, or a combination thereof.

7 and 8. (canceled)

9. The process of claim 1, wherein the alcohol is selected from the group consisting of an ethanol, a 1-propanol, a 1-butanol, a 1-pentanol, a 1-hexanol, a 1 ,2-propanediol, and mixtures thereof.

10. The process of claim 1, wherein the alcohol comprises a methanol, an ethanol, a butanol or a 1,2-propanediol.

11. The process of claim 1, wherein the heating is below the temperature of polymerization of the caprolactam.

12. A process for the synthesis of an ε-caprolactam, the process comprising:
(A) heating a salt of a lysine in a solvent comprising an alcohol, at a temperature of about 235° C. to about 320° C., to produce an α-amino-ε-caprolactam; and
(B) deaminating the α-amino -ε-caprolactam produced in (A) by a method comprising contacting it at least once with a deamination reagent or catalyst at a temperature below the freezing point of water, to produce the ε-caprolactam.

13. The process of to claim 12, wherein the lysine is an L-lysine.

14. The process of claim 12, wherein the temperature in (B) is from about −5° C. to about −20° C.

15. The process of claim 12, wherein the process further comprises a step (C) comprising washing the ε-caprolactam, produced by the deaminating (B), using a solvent wash.

16. The process of claim 15, wherein the washing solvent comprises a mixture of water and alcohol, or the washing solvent comprises a water.

17. (canceled)

18. The process of claim 12, further comprising purifying the ε-caprolactam.

19. The process of claim 18, wherein the purifying is by sublimation.

20. The process of claim 12, wherein the alcohol has from 2 to 6 carbons.

21. The process of claim 12, wherein the deaminating (B) employs a potassium hydroxide and a hydroxylamine-O-sulphonic acid.

22. The process of claim 1, wherein the alcohol is a methanol.

23. The process of claim 1, wherein the mass ratio of alcohol to lysine is between about 0.1:1 to about 100:1.

24. The process of claim 1, wherein the reactor system pressure is between about 1,000 psi and about 3,500 psi.
